(19) United States
(12) Patent Application Publication
(10) Pub. No.: US 2025/0108168 A1
(43) Pub. Date: Apr. 3, 2025

(54) MEDICAL DELIVERY ASSEMBLY

(71) Applicant: Koska Family Limited, East Sussex (GB)

(72) Inventors: Max Kingsley HANNON, London (GB); Eric Dwyer GIBNEY, West Chester, PA (US)

(21) Appl. No.: 18/979,090

(22) Filed: Dec. 12, 2024

Related U.S. Application Data
(63) Continuation of application No. PCT/US23/25123, filed on Jun. 13, 2023.
(60) Provisional application No. 63/351,810, filed on Jun. 13, 2022.

(51) Int. Cl. A61M 5/28; A61M 5/32; A61M 5/34

(52) U.S. Cl. CPC A61M 5/282; A61M 5/288; A61M 5/3202; A61M 5/346; A61M 2205/583

(57) ABSTRACT

A pre-filled medical delivery assembly assembled and configured to allow delivery of a single dose of a therapeutic agent (e.g., vaccine, drug, medicament, etc.) from a Blow-Fill-Seal (BFS) vial to a patient. The delivery assembly generally includes a modular design consisting of separately constructed components cooperatively arranged and coupled to one another. In accordance with some embodiments, the medical delivery assembly comprises a hub connector that includes at least one alignment track on an interior portion thereof, configured to receive a corresponding wing of a BFS vial which it is designed to couple with.

### CROSS-REFERENCE TO RELATED APPLICATIONS

The present application claims benefit and priority to, and is a non-provisional of, PCT Application No. PCT/US23/25123, filed on Jun. 13, 2023 and entitled MEDICAL DELIVERY ASSEMBLY; which PCT Application claims priority to U.S. Provisional Patent Application No. 63/351,810, filed on Jun. 13, 2022, and entitled “NEEDLE HUB FOR PRE-FILLED MEDICAL DELIVERY ASSEMBLY.” The entire contents of each of these Applications are hereby incorporated by reference herein for all purposes.

### BACKGROUND

Applicant has previously developed various single-use, single-dose, pre-filled and/or disposable medical injection devices and systems, such as ones utilizing a fluid container or vial produced in accordance with a Blow-Fill-Seal (BFS) manufacturing process. The vial is mated to one or more components including an administration member for injecting a fluid (e.g., a pharmacological agent) from the BFS vial into a patient. Commonly assigned U.S. patent application Ser. No. 17/849,780 entitled “SYSTEMS AND METHODS FOR FLUID DELIVERY” is one example of some embodiments of such injection devices and systems. These injection devices and systems may benefit from an ability to couple to a wider variety of medical devices, but configuring BFS vials for such connections has proven difficult due to the manufacturing limitations inherent in BFS manufacturing processes. In the case of small form factor BFS vials, the ability to deliver the entirety of the single-dose stored in the BFS vial may also be of concern due to the nature of the couplings.

### SUMMARY

According to some embodiments of the invention(s) described herein, a connector assembly for coupling to a compressible (e.g., radially compressible) vial containing a fluid agent comprises a hub connector defining a longitudinal axis and having an internal chamber for at least partial reception of a neck of the vial, a delivery member secured to the hub connector with the delivery member defining a subject end configured for penetrating a subject and an opposed vial end configured for penetrating the neck of the vial and at least one alignment track extending along the longitudinal axis and configured to receive a corresponding portion of the vial that extends outward from a body of the vial (e.g., at least one wing or other longitudinally oriented protrusion) of the vial in secured relation therewith, to thereby couple the hub connector to the vial to enable dispensing of the fluid agent through the delivery member. In some embodiments, the vial end of the delivery member is configured to penetrate the neck of the vial during coupling of the hub connector to the vial.

According to some embodiments, the at least one alignment track functions to guide the administration member such that it penetrates a desired area or position of the vial in a reliable and/or consistent fashion and/or orientation (e.g., sufficiently on-center). According to some embodiments, the at least one alignment track is dimensioned to at least partially compress at least a portion of the vial (e.g., the at least one wing or other protrusion) of the vial. First and second alignment tracks for receiving and compressing respective wings of the vial may be provided. In some embodiments, the first and second alignment tracks are disposed in diametric opposed relation. In some embodiments, each of the alignment tracks includes an enclosed channel segment and an open channel segment, wherein the wings of the vial are compressed at least within the enclosed channel segments of the alignment tracks when the hub connector is coupled to the vial. According to some embodiments, the hub connector includes an outer wall defining axial slots therein with the open channel segments of the alignment tracks comprising the axial slots and where segments of the wings of the vial are received within the axial slots when the hub connector is coupled to the vial.

According to some embodiments, the hub connector includes at least one internal axial rib. The at least one internal axial rib is configured to engage at least the neck of the vial to stabilize the vial relative to the vial end of the delivery member during coupling of the hub connector to the vial. In some embodiments, a plurality of internal axial ribs is provided. The internal axial ribs are configured to engage at least a collar segment of the neck of the vial.

In some embodiments, the hub connector includes at least one retention ledge with the retention ledge being configured to engage the neck of the vial to facilitate retention of the hub connector and the vial in coupled relation. In some embodiments, the retention ledge is configured to engage a collar segment of the neck of the vial. In some embodiments, the hub connector defines a window adjacent the retention ledge. The window, in some embodiments, enables viewing of the vial. In some embodiments, the window is configured to at least partially accommodate the collar segment. In some embodiments, the hub connector includes diametrically opposed retentions ledges configured to engage the collar segment of the vial. In some embodiments, the hub connector includes internal chamfered surfaces leading to the retention ledges. The chamfered surfaces are configured to facilitate passage of the collar segment of the vial through the hub connector during coupling of the hub connector to the vial. A cover may be positionable over at least the subject end of the delivery member and releasably coupled to the hub connector.

According to some embodiments, a connector assembly for coupling to a compressible vial containing a fluid agent comprises a hub connector defining a longitudinal axis and having leading and trailing ends with the hub connector defining an open internal chamber for at least partial reception of a neck of the vial, a pair of alignment tracks extending from the trailing end of the hub connector and disposed in diametric opposed relation with each alignment track including an enclosed channel segment, one or more internal axial ribs extending along the longitudinal axis within the internal chamber of the hub connector and a needle secured to the hub connector defining a subject end configured for application to a subject and an opposed vial end configured for penetrating the neck of the vial. During mounting of the compressible vial to the hub connector, the neck of the vial is received within the internal chamber of the hub connector and the outwardly depending opposed wings of the vial are received within the enclosed channel segments of the alignment tracks. The compressible vial is longitudinally advanced relative to the hub connector with respect to the longitudinal axis such that the enclosed channel segments compress the depending opposed wings of the vial to couple the compressible vial to the hub connector. In concert therewith, the internal axial ribs stabilize the neck of the vial relative to the vial end of the needle to enable penetration and access to the fluid agent.

In some embodiments, the hub connector includes an internal ledge. The internal ledge engages a collar segment of the neck of the vial during longitudinal advancing movement of the compressible vial whereby, upon clearing the internal edge, the collar segment of the vial is retained within the hub connector through engagement with the internal ledge. In embodiments, the hub connector includes a window adjacent the internal ledge.

According to some embodiments, a medical delivery assembly comprises a vial component including an outer vial wall defining a longitudinal axis and having a compressible (e.g., radially compressible) internal chamber for storing a fluid agent, a vial neck extending form the outer vial wall and one or more wings depending outwardly from the vial wall and extending along the longitudinal axis. The medical delivery assembly further comprises a connector component including a hub connector having an internal chamber for at least partial reception of the vial neck of the vial, an administration member secured to the hub connector and defining a subject end and an opposed vial end configured for penetrating the neck of the vial and one or more alignment tracks configured to receive the one or more corresponding wings of the vial in compressed relation therewith, to thereby couple the connector to the vial to enable dispensing of the fluid agent through the administration member.

In some embodiments, the vial includes first and second wings depending outwardly from the outer vial wall and the hub connector includes first and second alignment tracks configured to respectively receive the first and second wings of the vial in compressed relation therewith. In some embodiments, the first and second wings are disposed in diametric opposed relation to each other and the first and second alignment tracks are disposed in diametric opposed relation. In some embodiments, each of the first and second alignment tracks includes an enclosed channel segment whereby the first and second wings of the vial are compressed within the enclosed channel segments when the hub connector is coupled to the vial.

In some embodiments, the first and second wings define a wing dimension transverse to the longitudinal axis and wherein the enclosed channel segments of the first and second alignment tracks define a channel dimension transverse to the longitudinal axis. The channel dimension is less than the wing dimension such that the first and second wings are compressed within the enclosed channel segments of the first and second alignment tracks. In some embodiments, each of the first and second alignment tracks includes an open channel segment wherein segments of the first and second wings of the vial are received within the open channel segments of the first and second alignment tracks when the hub connector is coupled to the vial.

In some embodiments, the neck of the vial includes a collar segment. The collar segment is engaged by a retention ledge of the hub connector when the hub connector is coupled to the vial. In some embodiments, the hub connector defines a window adjacent the retention ledge. The window may be configured to at least partially accommodate the collar segment. In some embodiments, the hub connector includes an internal chamfered surface leading to the retention ledge with the chamfered surface being configured to facilitate passage of the collar segment of the vial through the hub connector during coupling of the hub connector to the vial. The hub connector may include multiple retentions ledges.

In some embodiments, the hub connector includes one or more slots. The one or more slots are configured to receive the first and second wings of the vial in a first rotational orientation of the vial relative to the hub connector, wherein, in the first rotational orientation, the opposed vial end of the administration member is spaced from the vial. The first rotational orientation corresponds to a transport condition of the medical delivery assembly. In some embodiments, the vial is removable from the hub connector to disengage the first and second wings from the one or more slots of the hub connector to enable the hub connector and the vial to assume a second rotational orientation, wherein, in the second rotational orientation, the first and second wings are receivable relative to the first and second alignment tracks. The second rotational orientation corresponds to an operative condition of the medical delivery assembly.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1 is an exploded perspective view of a medical delivery assembly illustrating a radially compressible vial, a hub connector with an administration member and a cover according to some embodiments;

FIG. 2 is a perspective view of a medical delivery assembly according to some embodiments;

FIG. 3 is a side cross-sectional view of a medical delivery assembly according to some embodiments;

FIGS. 4A-4C are side elevation, top plan and side cross-sectional views of the vial of a medical delivery assembly according to some embodiments;

FIGS. 5A and 5B are perspective views of the hub connector of a medical delivery system according to some embodiments;

FIGS. 5C, 5D and 5E are side elevation, side cross-sectional and top cross-sectional views respectively of the hub connector of a medical delivery assembly according to some embodiments;

FIG. 6 is an exploded perspective view of the hub connector and the administration member of a medical delivery assembly according to some embodiments;

FIGS. 7A and 7B are side elevation and side cross-sectional views of the cover of a medical delivery assembly according to some embodiments;

FIGS. 8-10 are side cross-sectional views illustrating an exemplary methodology of use of the medical delivery assembly according to some embodiments;

FIG. 11 is an axial cross-sectional view taken along the lines 11-11 of FIG. 10 of the medical delivery assembly according to some embodiments;

FIG. 12 is an exploded perspective view a medical delivery assembly illustrating a radially compressible vial, a hub connector with an administration member and a cover according to some embodiments;

FIG. 13 is a partial perspective view illustrating the medical delivery assembly in a transport condition according to some embodiments; and

FIG. 14 is a side cross-sectional view of the medical delivery assembly in the transport condition according to some embodiments.

### DETAILED DESCRIPTION

### I. Introduction

Embodiments of the present invention(s) provide systems and methods for pre-filled medical delivery assemblies that overcome drawbacks of current delivery devices and methods. For example, the pre-filled medical delivery assemblies of some embodiments may include a Blow-Fill-Seal (BFS) vial or bottle coupled to one or more specialized collars, assemblies and/or connectors that facilitate coupling of an administration member (e.g., a needle) to the BFS vial. Utilization of such systems that employ BFS bottles and specialized connection assemblies described herein may be advantageous and may address various shortcomings of previous systems.

Embodiments described herein provide systems and methods for medical delivery assemblies that overcome drawbacks of current delivery devices and methods. For example, the medical delivery assemblies of some embodiments may include a compressible vial or bottle pre-filled with a fluid agent and a hub connector which is couplable to the vial at a procedural site. The hub connector includes an administration member or delivery member such as a cannulated needle coupled thereto. The hub connector includes features which facilitate alignment of the vial relative to the hub connector during coupling of the components and piercing of the vial with the needle. The hub connector also stabilizes the vial to eliminate leakage of the fluid agent during coupling and/or use in delivering the fluid agent from the compressible vial into or relative to the patient.

In some embodiments, the vial is a radially compressible vial fabricated using Blow-Fill-Seal (BFS) technology. BFS technology is a manufacturing technique used to produce small volume (e.g., 0.1 mL) and large volume (e.g., 500 mL+) liquid-filled containers. BFS vials may, for example, offer a less expensive alternative to typical vials or devices created via other manufacturing techniques. In some embodiments, BFS vials (e.g., due to the nature of the BFS manufacturing process) may not require separate sterilization (e.g., and may accordingly be compatible with a wider array of fluid agents) and may provide enhanced production rates of approximately thirty thousand (30,000) sterile/aseptic units per hour. In some embodiments, these advantages may come with attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)-for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989). Accordingly, there exists a need for a design of a hub connector that can be mated with a BFS vial in a manner that overcomes such drawbacks by, for example, not relying on overly precise geometry or manufacturing tolerances in order to provide a sufficient coupling mechanism.

In accordance with some embodiments, the hub connector described herein mates to, or couples with, the soft plastic of the BFS vial without substantively compressing or squeezing the compressible portion of the BFS vial over which it sits, and without requiring an overly precise geometry or shape of the BFS vial. One aspect of the mating or coupling elements of the hub connector described herein reduces the likelihood that any fluid agent within the BFS vial will leak during puncturing of the needle of the hub connector but prior to a user squeezing the vial to inject the contents into the patient. Another aspect of the coupling elements of the hub connector described herein allows for sufficient tolerances in the geometry and shape of the BFS vial, such that there is some flexibility in the ability of the hub connector to fit over a neck portion of the BFS vial.

### II. Pre-filled Medical Delivery Assemblies

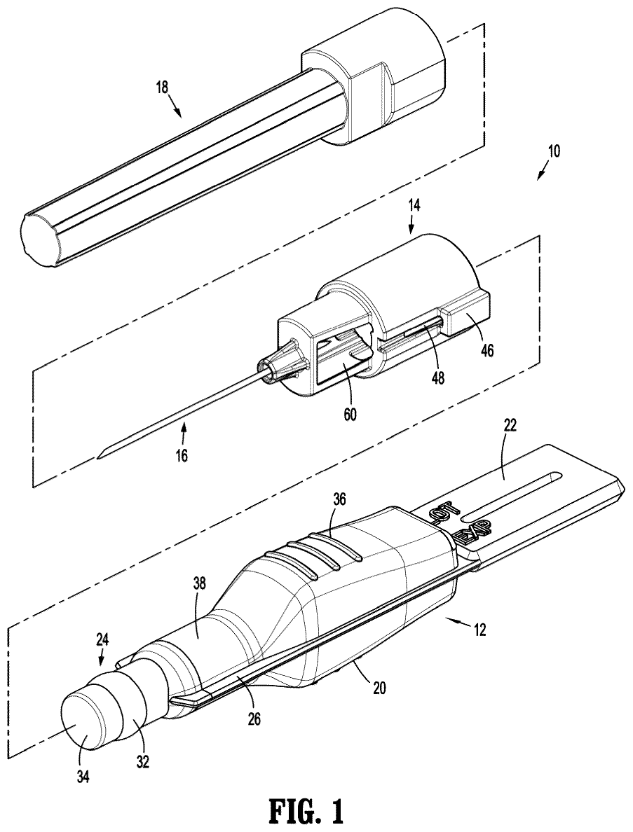

Referring now to FIGS. 1-3, a medical delivery assembly according to some embodiments is illustrated. The medical delivery assembly 10 may include, for example, a compressible BFS cartridge or vial 12, a hub connector 14 with an attached delivery or administration member 16 (for example, a cannulated needle) and a cover 18 which is positionable over the administration member 16. In some embodiments, the medical delivery assembly 10, including each of the components, may be provided to a user in a single package. In some embodiments, components of the medical delivery assembly 10 may be provided individually to the user. For example, and without limitation, the hub connector 14, attached administration or delivery member 16 and needle cover 18, may be provided and packaged as an assembled modular unit of the medical delivery assembly 10 separate from the vial 12. The vial 12 may be provided as one vial component of a plurality of attached vials of, for example, a vial cartridge system (not shown). In some embodiments, the vial 12, hub connector 14, administration member 16 and cover 18 may be packaged together.

For reference purposes, the medical delivery assembly 10 includes a leading or distal end adjacent the administration member 16 which is the end closest to the patient during administration of the fluid agent, and a trailing or proximal end adjacent the vial 12 which is the end remote from the patient. Thus, the use of the term “leading,” “leading end” or “leading direction” is to be interpreted as the direction generally moving toward the administration member 16 and the term “trailing,” “trailing end” or “trailing direction” is to be interpreted as the direction generally moving toward the vial 12 away from the administration member 16.

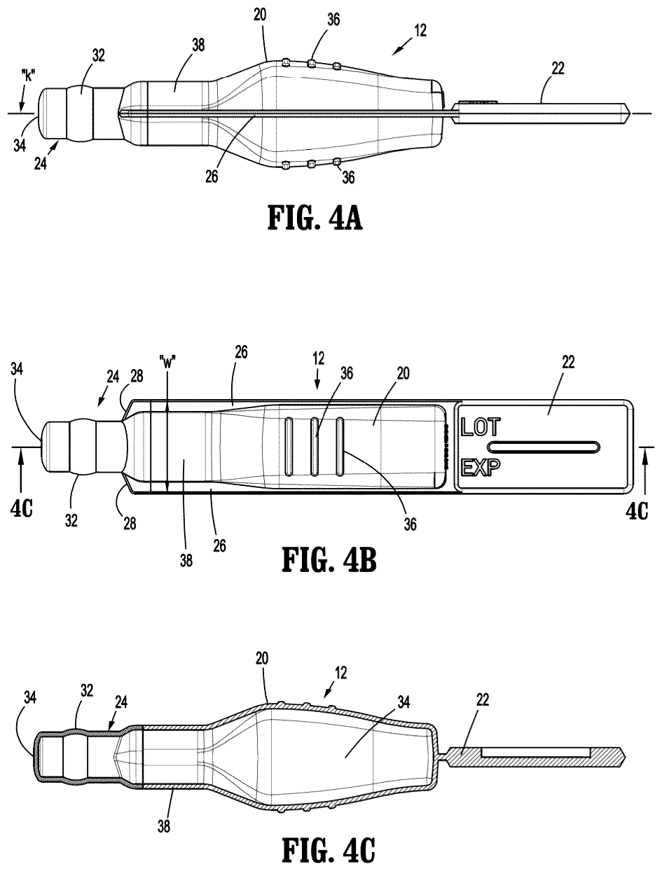

With reference FIGS. 4A-4C, in view of FIGS. 1-3, the vial 12 will be discussed. The vial 12 may be fabricated using BFS technology. During the BFS manufacturing process, the vial 12 is formed, filled, and sealed in a continuous process without material human intervention, in a sterile enclosed area inside a machine. In particular, a rotary-style BFS manufacturing process involves multiple steps in which a pharmaceutical-grade plastic resin is vertically heat extruded through a circular opening (not shown) to form a hanging tube called a parison (not shown). This extruded tube is then enclosed within a two-part mold (not shown), and the tube is cut above the mold. The mold is transferred to the filling zone, or sterile filling space, where filling needles (mandrels; not shown) are lowered and used to inflate the plastic to form the container/vial 12 within the mold. Following the formation of the vial 12, the mandrel is used to fill the container with liquid or fluid agent (not shown). Following filling, the mandrels are retracted and a secondary top mold seals the container. Alternatively, a vial 12 may be manufactured using a shuttle-style BFS manufacturing process. In either type of BFS manufacturing process, the manufacturing actions take place inside a sterile chamber (not shown) inside the machine. The product (the BFS vial 12, filled with a fluid agent and sealed) is then discharged to a non-sterile area for labeling, packaging and distribution. In some embodiments, the compressible vial 12 includes a single dose of the fluid agent or, optionally, may include multiple doses.

In some embodiments, the vial 12 includes a compressible vial body 20 defining a longitudinal axis “k,” a vial handle 22 extending from one end of the vial body 20 and a vial neck 24 extending from the other end of the vial body 20. The vial body 20 includes one or more wings 26 depending radially outwardly with respect to the longitudinal axis “k.” In some embodiments, first and second wings 26 are provided and arranged in diametric opposed relation. The first and second wings 26 are narrow in cross-section defining a thickness, in some embodiments, less than one (1) millimeter. In some embodiments, the first and second wings 26 define a wing dimension “w” orthogonal to the longitudinal axis “k” ranging from about to about 11.7 millimeters (mm) to about 12.7 millimeters (mm), or about 12.1 millimeters (mm) or, in some embodiments, about 12.4 millimeters (mm). Other dimensions are also contemplated. The wings 26 may, in some embodiments, include tapered leading wing segments 28 which terminate adjacent the vial neck 24. The leading wing segments 28 may be arranged at an oblique angle to facilitate insertion within the hub connector 14. The first and second wings 26 may be more rigid or stiff relative to the vial body 20.

The vial handle 22 may be planar and include indicia or an indicator (such as an RFID chip) representative of the type, lot, expiration date of the fluid agent contained within the vial body 20. The vial neck 24 may comprise an annular collar 32 disposed at an intermediate location of the vial neck 24 and extending outwardly from the vial neck 24, and a penetrable leading vial end face or seal 34. In some embodiments, the annular collar 32 may be a donut-shaped protrusion extending radially outwardly from the remainder of the vial neck 24. In some embodiments, the annular collar 32 may be a partial annular structure or segment, or include multiple structures. In some embodiments, the annular collar 32 may be any structure depending outwardly from the vial neck 24. In some embodiments, the annular collar 32 may be configured to compress radially inwardly during coupling of the vial 12 to the hub connector 14 and return outwardly to its normal condition when coupling of the components is effected. In some embodiments, the annular collar 32 assists in stabilizing the vial 12 relative to the hub connector 14 during coupling and penetration of the administration member 16 through the vial end face 34 of the vial neck 24, thereby minimizing the potential of undesired leakage of the fluid agent from the vial body 12. The annular collar 32 may also or alternatively serve to stabilize the vial 12 relative to the hub connector 14 during manipulation and administration of the fluid agent to the subject or patient with the administration member 16. In some embodiments, the annular collar 32 may function or assist in functioning to secure the vial neck 24 and the vial 12 to the hub connector 14. In some embodiments, the annular collar 32 is included on the vial neck 24, but it does not play any functional role in securing the vial neck 24 and the vial 12 to the hub connector 14. In some embodiments, the annular collar 32 does not compress during insertion within the hub connector 12. In some embodiments, the annular collar 32 is omitted from/not included on the vial neck 24 or may be replaced with a feature of a different geometry (e.g., tabs or one or more other protrusions along the same portion of the vial, rather than the donut-like shape that extends around the full circumference of the vial as represented in the FIG. 1).

According to some embodiments, the vial body 20 defines an internal vial reservoir 34 extending to the vial neck 24 which is at least partially filled with a fluid agent (not shown). The vial body 20 may be radially compressible in whole, or in part, to compress the internal vial reservoir 34 and pressurize and/or eject the fluid agent. The vial body 20 further includes one or more gripping ribs 36 on its external surface to facilitate engagement by the user. In some embodiments, gripping ribs 36 are provided on each side of the vial body 20. In some embodiments, gripping ribs 36 are omitted. Disposed between the vial body 20 and the vial neck 24 is a vial mount 38 which is at least partially received within the hub connector 14. In some embodiments, the vial mount 38 is correspondingly dimensioned to axially align the vial 12 with the hub connector 14 during coupling of the components. The vial mount 38 defines a dimension less than the dimension of the vial body 20. The first and second wings 26 depend outwardly from the vial mount 38 to engage cooperative mating structure within the hub connector 14 (e.g., alignment tracks 46, described below).

According to some embodiments, the internal vial reservoir 34 includes one unrestricted chamber. In some other embodiments, the internal vial reservoir 34 includes two adjoining chambers or portions, for example, one chamber within the vial body 20 and a second chamber within the vial mount 38 or two chambers within the vial body 20. In some embodiments, the internal vial reservoir 34 is unrestricted such that fluid may generally and/or freely pass between the chamber within the vail body 20 and the chamber within the vial mount 38. In some other embodiments, a juncture, restriction, valve, and/or passage (not separately labeled and collectively referred to as a “restriction feature”) may be disposed between the respective chambers of the vial body 20 and the vial mount 38. The restriction feature may restrict flow such that the fluid may readily enter the chamber within the vial mount 38 from the chamber within the vial body 20, but may not readily return to vial body 20.

The vial 12 may be transparent in whole, or in part. In some embodiments, at least the vial neck 24 of the vial 12 is transparent to enable viewing of the fluid agent before, during and/or subsequent to administration to the subject. This feature enables the user to determine if the status of the volume of fluid agent, for example, its location, whether it is exhausted or if some fluid agent remains within the vial 12.

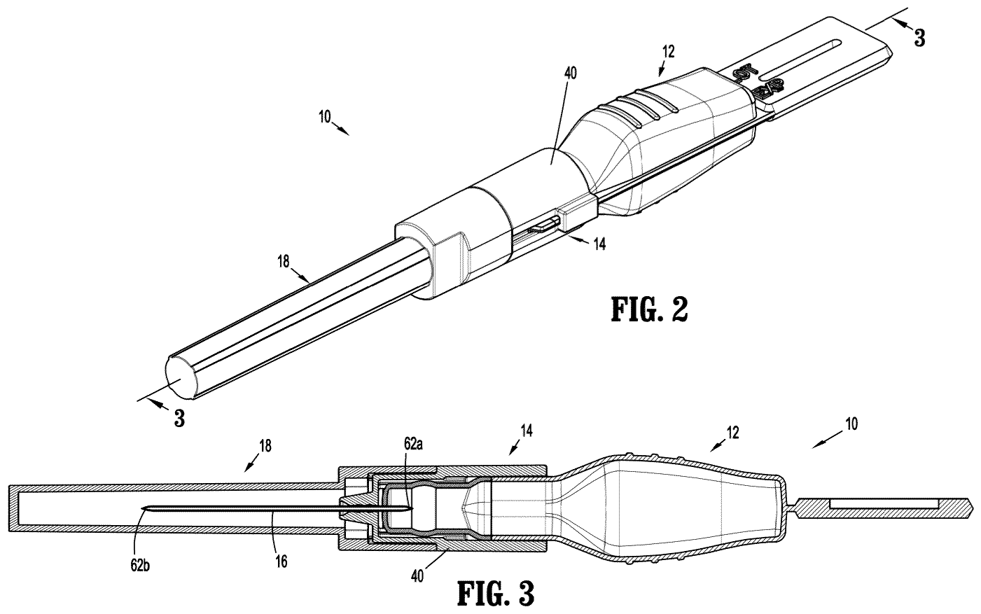

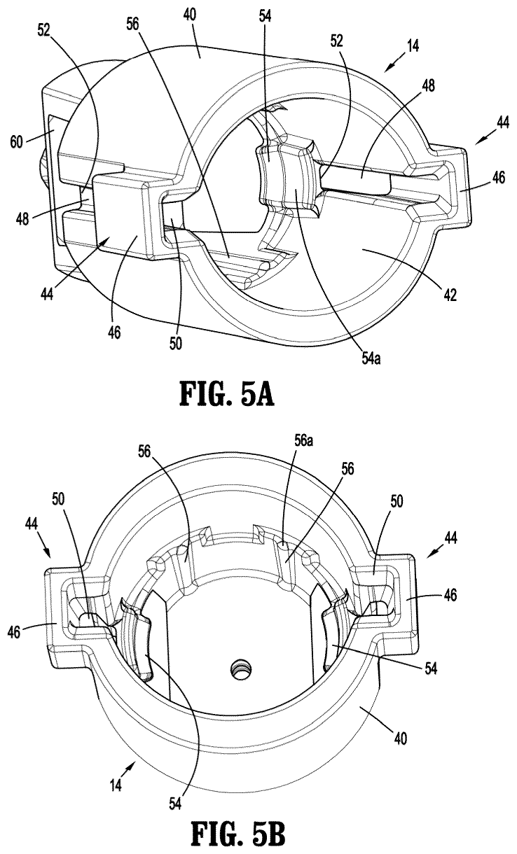

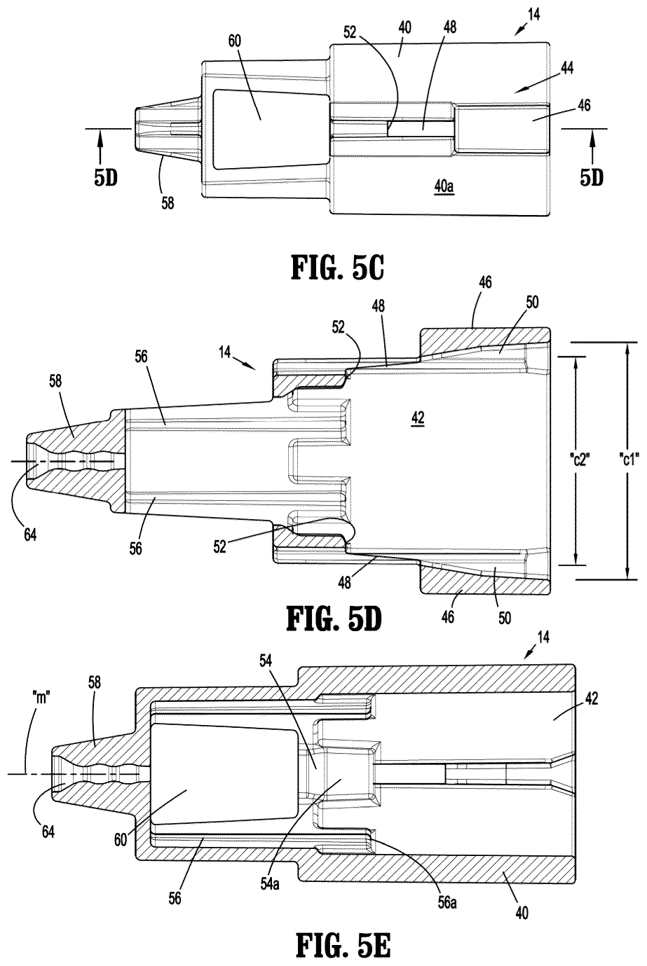

Referring now to FIGS. 5A-5E, in view of FIGS. 1-3, the hub connector 14 will be discussed. The hub connector 14 includes a connector housing 40 defining a longitudinal axis “m” which is coincident with the longitudinal axis “k” of the vial body 20 in the coupled relation of the hub connector 14 and the vial 12 depicted in FIGS. 2 and 3. The connector housing 40 defines an internal housing chamber 42 for reception of the vial neck 24 and the vial mount 38 of the vial 12. The connector housing 40 further includes one or more alignment tracks 44 configured for reception of at least portions of the one or more wings 26 of the vial 12. In some embodiments, the connector housing 40 includes first and second alignment tracks 44 in diametric opposed relation for respective reception of the first and second wings 26 of the vial 12. Each alignment rack 44 includes an enclosed channel segment 46 leading to an open channel segment 48. The enclosed channel segments 46 each define a longitudinal rail or groove 50 for reception of the first and second wings 26 of the vial 12. In some embodiments, the enclosed channel segments 46 are arranged to define longitudinal grooves 50 that generally taper inwardly toward the leading end of the hub connector 14 relative to the longitudinal axis “m.” (FIG. 5D). In some embodiments, the taper is gradual or linear. In some embodiments, the taper is non-linear. In some embodiments, the enclosed channel segments 46 define an internal dimension “c1” adjacent the trailing end of the connector housing 40 which is greater than the internal dimension “c2” adjacent the open channel segments 44. The internal dimension “c1” may generally correspond to, or be slightly greater than, the wing dimension “m” or span defined by the wings 26 of the vial 12 to enable initial reception of the wings 26 within the longitudinal grooves 44. The internal dimension “c2” is less than the wing dimension “w” defined across the wings 26. In some embodiments, the internal dimension “c2” ranges from about 11.0 millimeters to about 11.5 millimeters (mm), or is about 11.33 millimeters (mm). Thus, during coupling and insertion of the vial 12 to the hub connector 14, the wings 26 are compressed (e.g., radially inward) as they traverse the longitudinal grooves 50 of the enclosed channel segments 46. The compression of the wings 26 facilitates retention of the vial 12 relative to the connector housing 40 of the hub connector 14 by at least forming an interference fit between the components as will be discussed hereinbelow. In some embodiments, the longitudinal grooves 50 do no taper but define the same or similar internal dimension throughout the lengths of the enclosed channel segments 46.

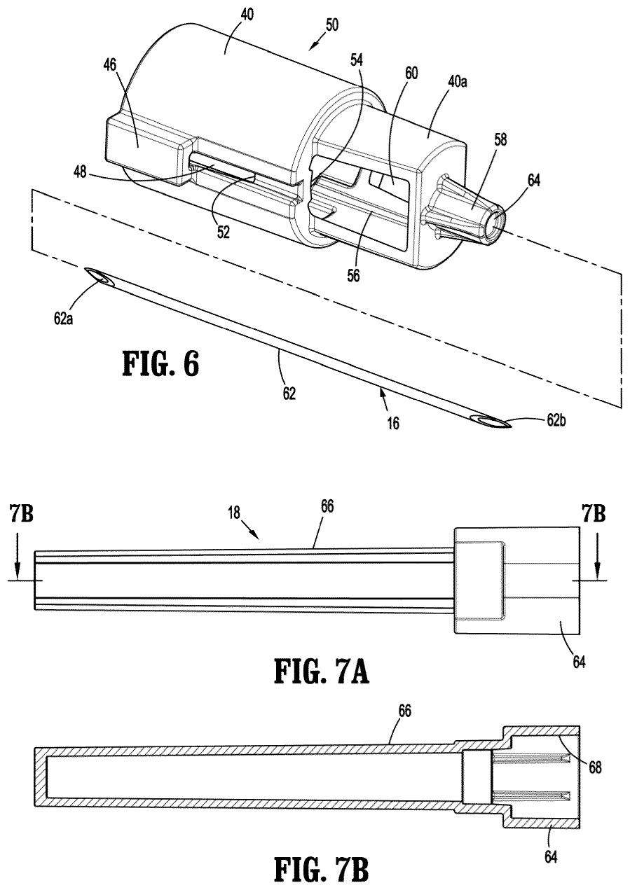

With continued reference to FIGS. 5A-5E and FIG. 6, the open channel segments 48 of the alignment tracks 44 may continue from the enclosed channel segments 42. In some embodiments, the open channel segments 48 are defined by slots formed in the housing wall 40a of the connector housing 40. The open channel segments 48 receive at least the leading portions of the wings 26, including the tapered leading wing segments 28 of the vial 12, when the vial 12 is coupled to the hub connector 14. The open channel segments 48 each terminate in a stop 52, which, in some embodiments, is defined by an internal wall of the connector housing 40. In some embodiments, the stops 52 limit traversing leading movement of the wings 36 within the alignment tracks 44.

The connector housing 40 further includes one or more retention tabs or ledges 54 within the internal housing chamber 42 depending inwardly relative to the longitudinal axis “m” of the hub connector 14. In some embodiments, two retention ledges 54 are provided and disposed in diametric opposed relation. The one or more retention ledges 54 are configured to engage the annular collar 32 of the vial neck 24 during coupling of the vial 12 to the hub connector 14, and in some embodiments, assist in retaining the vial 12 in coupled relation with the hub connector 12. The opposed retention ledges 54 may define an internal dimension less than the outer dimension of the annular collar 32 of the vial 12 whereby during advancing movement of the vial neck 24 within the connector housing 40, the annular collar 32 deforms to pass the retention ledges 54 and then assumes its normal outward condition when released beyond the retention ledges 54. The retention ledges 54 may include internal chamfered surfaces 54a configured to facilitate passage of the collar segment 32 of the vial 12 through the connector housing 40 during coupling of the hub connector 14 to the vial 12.

As best depicted in FIGS. 5B and 5D, the hub connector 14 may comprise one or more internal axial ribs 56 disposed within the internal housing chamber 42 of the connector housing 40. The internal axial ribs 56 may start at an intermediate location, for example, near the stops 52, and extend to the hub nose 58 of the connector housing 40. The one or more internal axial ribs 56 are configured to engage at least the collar segment 32 of the vial neck 24 of the vial 12 to stabilize the vial 12 within the connector housing 40. In some embodiments, the connector housing 40 includes a plurality of internal axial ribs 56 arranged in radial spaced relation within the internal housing chamber 42. In some embodiments, the one or more internal axial ribs 56 includes first and second pairs of internal axial ribs 56 arranged in diametric opposed relation, for example one set at about the 5 o'clock and 7 o'clock positions and a second set at the 11 o'clock and 1 o'clock positions. The internal axial ribs 56 may include lead-in tapered segments 56a to facilitate insertion and advancement of the vial neck 24. The internal axial ribs 56 are configured and dimensioned to stabilize the vial neck 24 and the vial 12 during coupling and use of the assembly 10. In some embodiments, the internal axial ribs 56 provide multiple points or lines of contact with the periphery of the vial neck 24 and/or the annular collar 32 of the vial 12 to stabilize the vial 12 both during and subsequent to coupling to the hub connector 14. In some embodiments, the internal axial ribs 56 engage the annular collar 32 of the vial neck 24 to provide four points or lines of contacts distributed about the periphery of the annular collar 32 to stabilize the vial neck 24 within the connector housing 40. In some embodiments, the internal axial ribs 56 engage the annular collar 32 and/or the remaining portions of the vial neck 24.

As best depicted in FIGS. 5A, 5C and 5E, the connector housing 40 of the hub connector 14 may comprise one or more windows 60 defined in the connector housing 40 distal of the retention ledges 54 and generally longitudinally adjacent the one or more internal axial ribs 56. (See also FIG. 6). In some embodiments, the connector housing 40 includes two opposed windows 60. The one or more windows 60 may provide multiple functions. For example, the one or more windows 60 enable a user to visualize the vial neck 24 and/or the annular collar 32 of the vial 12 within the hub connector 14 to determine the status of the fluid agent within the vial 12. For example, the one or more windows 60 enable the user to determine the location of the fluid agent within the vial 12 and confirm that the fluid agent has moved from the valve body 20 to within the vial neck 24. The one or more windows 60 may therefore allow the user to avoid repeatedly having to “flick” or “whip” the vial 12 to move the fluid from the internal vial reservoir 36 to the vial neck 24. The one or more windows 60 also enable the user to visualize the location of the fluid agent relative to the leading face seal 34 of the vial neck 24 and thereby avoid causing any fluid agent to escape from the vial 12 when the administration member 16 punctures the leading face seal 34.

The one or more windows 60 also enables the user to visually inspect the vial 12 to verify that the fluid therein was successfully and completely administered to the patient, and that no fluid remains within the vial 12. This enables the user to visually determine that the complete dosage of the fluid in the vial 12 was delivered to patient, and that there is no wastage of the fluid contents thereof.

In addition, in some embodiments, the one or more windows 60 may accommodate at least portions of the annular collar 32 (or a collar or feature of an alternate geometry that protrudes from the same or similar portion of the vial 12) of the vial 12 when the vial 12 is mounted to the hub connector 14. This feature or arrangement minimizes potential of compressive forces being exerted on the annular collar 32 and the vial neck 24 by the hub connector 14 when the vial 12 is coupled to the connector housing 40, thereby reducing the likelihood of fluid agent being inadvertently dispensed from the vial 12, particularly, for example, when the assembly 10 is manipulated about the site. In some embodiments, no portion of the annular collar 32 extends within the one or more windows 60.

Referring now to FIG. 6, the administration member 16 will be described. In some embodiments, the administration member 16 is a delivery member and, in embodiments, may include a needle for at least one of subcutaneous, intramuscular, intradermal, and intravenous injection of the fluid agent into the patient. For ease of explanation and description, the figures and the description herein generally refer to the administration member 16 as a needle. However, it should be noted that, in other embodiments, the administration member 16 may include a nozzle (not shown) configured to control administration of the fluid agent to the patient. The nozzle may include a spray nozzle, for example, configured to facilitate dispersion of the fluid agent into a spray or a droplet nozzle configured to disperse the fluid agent in the form of one or more drops. In some embodiments, the administration member 16 is a cannulated needle 62 having a vial penetrating end 62a and an opposed patient penetrating end 62b. (see also FIG. 3) The cannulated needle 62 is secured within a longitudinal bore 64 of the hub nose 58 of the connector housing 40 through the use of conventional methodologies including without limitation adhesives, welding, thermal bonding or combinations thereof, and is axially aligned with the longitudinal axis “m” of the hub connector 14. The vial penetrating end 62a of the cannulated needle 62 is disposed within the interior of the connector housing 40 at a predetermined longitudinal location to pierce the vial end face seal 34 of the vial 12 when the vial 12 is coupled to the hub connector 14. Each of the penetrating ends 62a, 62b may include a chamfered edge leading to a penetrating point. Other arrangements are also contemplated. In some embodiments, in which, for example, the administration member 16 is to be used as a dispensing nozzle, the penetrating end 62b may be replaced with an atraumatic feature such as a nozzle to avoid penetration of the subject.

With reference to FIGS. 7A and 7B, the cover 18 includes a cover housing 64 and an elongated cover component 66 depending from the cover housing 64. The cover housing 64 includes inner wall portions defining an internal cover chamber 68 configured to fit over the cover mount segment 40a of the connector housing 40 whereby, for example, an interference with the exterior of the connector housing 40 and the inner wall portions of the cover housing 64 is established. The elongated cover component 66 encloses the administration member 16 during transport and prior to use. In some embodiments, the cover 18 remains connected to the connector housing 40 during coupling of the hub connector 14 and the vial 12.

As generally understood, the fluid or drug agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the vial 12, hub connector 14 and/or the cover 18.

According to some embodiments, the hub connector 14 and the cover 18 may be composed of a medical grade material. In some embodiments, the hub connector 14 and the cover 18 may be composed of a thermoplastic polymer or other “hard” plastic (e.g., greater than 80 on the Rockwell “R” scale), including, but not limited to, polybenzimidazole, acrylonitrile butadiene styrene (ABS), polystyrene, polyvinyl chloride, or the like. The administration member 16, in the form of a cannulated needle, may be formed of a suitable biocompatible metallic material.

In some embodiments, the pre-filled medical delivery assembly 10 may be advantageously manufactured (in mass quantities) in separate parts or portions, namely, at least the “soft” plastic BFS vial 12 and the “hard” plastic hub connector 14 and the cover 18 with such different plastic parts/portions being selectively coupled to administer a medication to a patient. In practice, for example, some or all of the following procedures may be followed to utilize the pre-filled medical delivery assembly 10 to administer a medication to a patient.

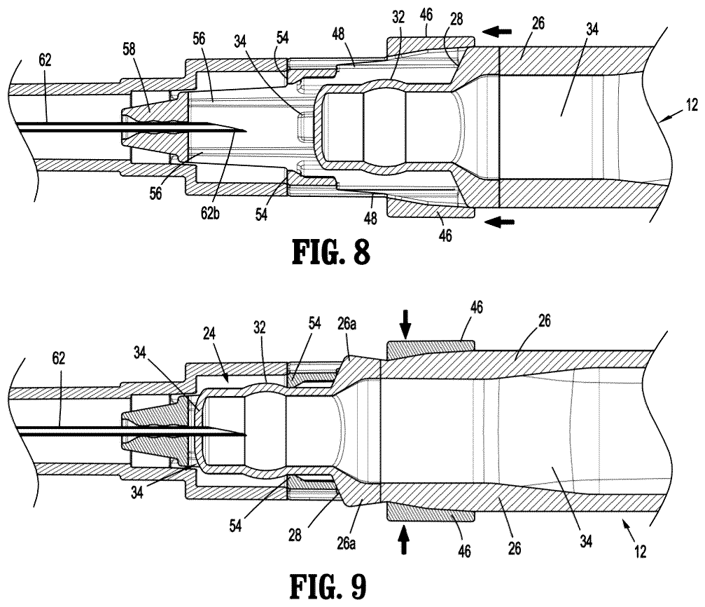

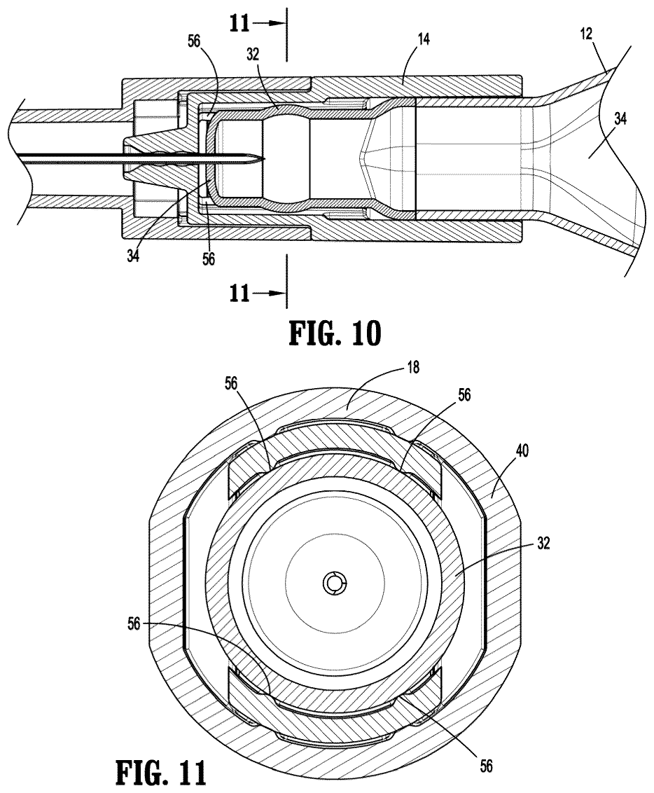

Referring now to FIGS. 8-10, one exemplative methodology for use of the medical delivery assembly 10 will be described. As noted hereinabove, in some embodiments, the vial 12 may be packaged independent of the rest of the assembly 10, and may be part of a supply of vials coupled to each other during for example, the BFS manufacturing process. When it is desired to assemble the medical assembly 10 for use, the vial 12 is removed from the supply of vials and the hub connector 12 with attached administration member 16 and cover 18 is removed from, for example, its packaging (not shown). The vial neck 24 of the vial 12 and the vial seal face 34 may be cleaned (e.g., utilizing an alcohol wipe). According to some embodiments, a seal (not shown) covering the opening into the internal housing chamber 42 of the connector housing 40 may be removed and/or punctured. With reference to FIG. 8, the vial 12 is aligned with the connector housing 14 (for example, to align the longitudinal axis “k” of the vial 12 with the longitudinal axis “m” of the hub connector 14). The vial neck 24 is introduced within the internal housing chamber 42 of the connector housing 14 with the wings 26 of the vial 12 received within the enclosed channel segments 46 of the alignment tracks 44. As noted, the enclosed channel segments define an internal dimension “c1” which may generally correspond to, or be slightly greater than, the wing dimension “m” defined by the wings 26 of the vial 12 to enable initial reception of the wings 26 within the longitudinal grooves 50 of the enclosed channel segments 46. The vial 12 and the hub connector 14 are moved longitudinally toward each other. For example, the vial 12 may be advanced within the hub connector 14 and/or the hub connector 14 may be pulled onto the vial 12. During this relative longitudinal movement, the wings 26 of the vial body 20 engage the (e.g., tapered dimensioning of) longitudinal grooves 50 of the enclosed channel segments 46 such that the wings 26 (for example, the outer surface portions of the wings 26) are compressed radially inwardly (represented by the directional arrows of FIG. 9) relative to the longitudinal axis “m” of the vial body 20 as the wings 26 traverse the longitudinal grooves 50. In addition, as the vial neck 24 and the vial 12 are advanced into the connector housing 40, the annular collar 32 of the vial neck 24 engages the internal chamfered surfaces 54a leading to the retention ledges 54. The annular collar 32 guided by the internal chamfered surfaces 54a deforms to enable passage through the retention edges 54 and then starts to assumes its normal outward condition as the vial 12 is continually advanced. In concert with the outward movement of the collar 32, the collar 32 engages the internal axial ribs 56 extending within the connector housing 40 toward the hub nose 58. The internal axial ribs 56 engage the outer surface of the annular collar 32 to stabilize the annular collar 32, the vial neck 24 and the end face seal 34 to facilitate penetration of the end face seal 34 by the penetrating end 60a of the cannulated needle. (FIG. 10). The collar 32 of the vial 12 assumes its normal expanded condition beyond the retention ledges 54, and, in some embodiments, at least partially accommodated by the windows 60 of the connector housing 40. (FIG. 9). In some embodiments, when the vial 12 is coupled to the hub connector 14, the user may receive a tactile or noise indicator, for example, when the annular collar 32 passes the retention ledges 54, representative that the coupling is properly effected.

FIGS. 9 and 10 illustrate the vial 12 in coupled relation with the hub connector 14. In the coupled relation, the vial 12 is retained within the connector housing 40 via the compressive forces exerted by the enclosed channel segments 46 on the wings 26. More specifically, as noted above, the enclosed channel segments 46 define a minimum internal dimension “c2” which is less than the wing dimension “m” defined by the wings 26 of the vial 12. Thus, the wings 26 are compressed during traversing through the longitudinal grooves 50 of the enclosed channel segments 46. The degree of compression is sufficient to deform the wings 26 and establish a friction or interference fit between the enclosed channel segments 46 and the wings 26 to secure the wings 26 within the enclosed channels 46 thereby securing the vial 12 within the hub connector 14. In some embodiments, the tapered arrangement of the enclosed channel segments 46 with the deformed portions of the wings 26 also establishes a Morse taper-like coupling between the components. In some embodiments, as the leading end segments 26a of the wings 26 clear the enclosed channel segments 46 and enter the open channel segments 48 of the alignment tracks 44, the leading wing end segments 26a may also move toward their original outward condition (the shape prior to insertion within the enclosed channel segments 46). (FIG. 9) This deflection outward of the leading wing segments 26a into the open channel segments 48 may also cause engagement of portions of the wings 26 with the outer end faces 46b of the enclosed channel segments 46 to further retain the wings 26 within the enclosed channel segments 46. In some embodiments, the leading end segments 26a of the wings 26 do not deflect outwardly. In addition, in some embodiments, the vial 12 also may be prevented from decoupling relative to the connector housing 40 through potential engagement of the annular collar 32 of the vial 12 with the retention ledges 54 of the connector housing 40.

Furthermore, in some embodiments, with the vial 12 and the hub connector 14 in the coupled relation depicted in FIGS. 10 and 11, the outer surface of the annular collar 32 is engaged and stabilized by the internal axial ribs 56 of the connector housing 40. The internal axial ribs 56 stabilize the collar 32 and the vial neck 24 during penetration and subsequent to penetration. The stabilizing features minimize the potential for the vial neck 24 to move around or “jostle” relative to the connector housing 40 and the administration member 16/cannulated needle 62. This minimizes potential of fluid agent inadvertently dispensing about the needle, for example, between the needle and the opening formed in the seal end face 34 by the needle.

With the vial 12 coupled to the hub connector, the cover 18 may be removed. The administration member 16 or needle is applied to the subject. The vial body 20 is compressed to dispense the contents from the internal vial reservoir 36 through the vial neck 24 and the administration member 16. Anytime during the procedure, the user can view through the windows 60 of the connector housing 40 to determine the status and/or location of the fluid agent within the transparent vial 12 to ascertain that the dosage has been delivered or only partially delivered.

In some embodiments, the force required to effect the coupling between the vial 12 and the hub connector 14 is less than about 35 newtons (N). The force required to remove the vial 12 from the hub connector, for example, after use, may be greater than about 18 Newtons (N). Forces required to remove the cover 18 from the hub connector 14 may range from about 2 to 16 Newtons (N).

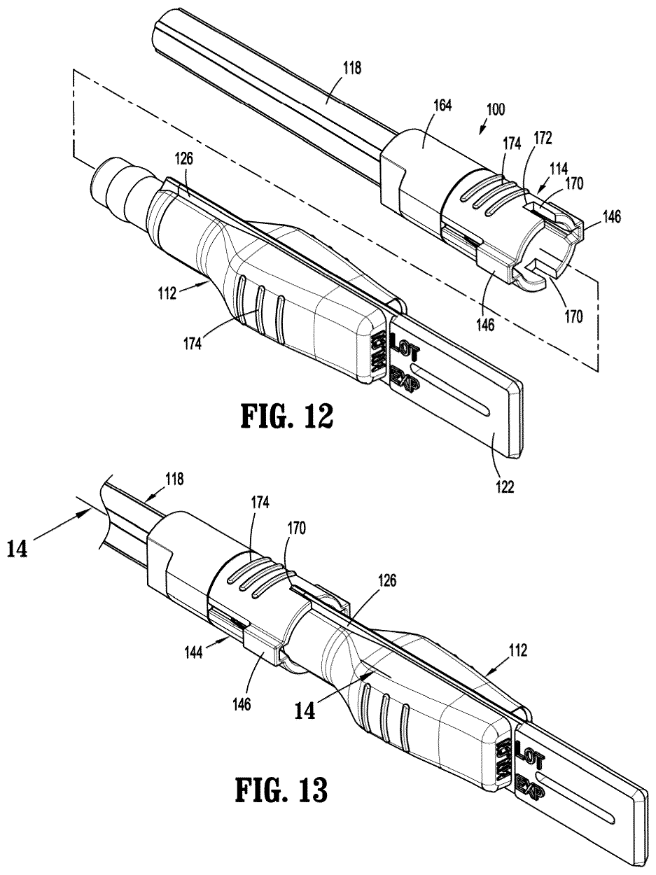

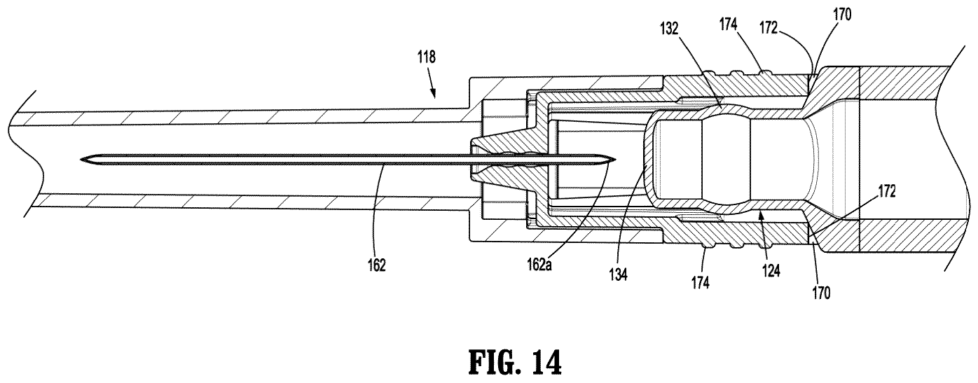

Referring now to FIGS. 12-14, one embodiment of the medical delivery assembly is illustrated. The medical delivery assembly 100 may be substantially similar to the medical delivery assembly 10 of FIGS. 1-11. For example, the vial 112, hub connector 114, administration member 116 and cover 118 may be substantially identical to the corresponding components of the medical delivery assembly 10. In some embodiments, the connector housing 140 of the hub connector 114 includes one or more axial slots 170 extending from the trailing end of the connector housing 140. In some embodiments, a pair of axial slots 170 is provided and arranged in diametric opposed relation. The axial slots 170 may be radially offset relative to the alignment tracks 144 of the hub connector by an angle of about 90 degrees. According to some embodiments, the one or more axial slots 170 enables coupling of the vial 112 and the hub connector 114 without causing piercing of the administration member 116 with the vial 112. This coupled configuration is therefore advantageous by enabling packaging/transporting all the components of the medical assembly 100 including the vial 112, the hub connector 114, the administration member 116 and the cover 118 as a single modular unit or component while avoiding accidental piercing/rupturing of the fluid end seal 134 of the vial 112 with the administration member 116. The length of the one or more axial slots 170 is selected such that the fluid end seal 134 of the vial 112 is spaced from the administration member 116 in the coupled transport arrangement of the vial 112 and the hub connector 114. For example, the wings 126 engage the stops 172 defined in the connector housing 140 coterminous with the axial slots 170. This prevents the trailing piercing end 162a of the cannulated needle 162/administration member 116 from reaching/contacting, and piercing/penetrating, the fluid end seal 134 of the vial 112 thereby avoiding unwanted dispensing and/or contamination of the fluid agent.

In some embodiments, the hub connector 116 further includes thumb placement indicators 174 disposed on one or both sides of the hub connector, e.g., arranged perpendicularly with respect to the viewing window(s) 160 and/or between two viewing windows 160. According to some embodiments, the thumb placement indicators 174 constitute raised or textured protrusions or ridges. The thumb placement indicators 174 are strategically configured and disposed on the hub connector 116 to facilitate a user's proper and effective handling of the pre-filled medical delivery assembly 100. In some embodiments, the thumb placement indicators 174 may be omitted.

When is time to use the medical assembly 100, the vial 112 may be withdrawn from the hub connector 114 and/or the hub connector 114 may be removed from the vial 112, and the components rotated through a predetermined angular rotation, for example, 90 degrees with respect to each other. In this position, the wings 126 of the vial 112 are aligned with the alignment tracks 144 of the hub connector 114. The vial 112 is then coupled to the hub connector 114 in the manner described hereinabove.

FIGS. 12-14 illustrate the transport condition of the medical assembly 100. In the transport condition, the vial 112 and the hub connector 114 are arranged at a first rotational orientation. The operative condition is represented in FIGS. 2 and 3. In the transport condition, the vial 112 and the hub connector 114 are arranged at a second rotational orientation.

### III. Multiple Inventive Embodiments Disclosed

Multiple inventive embodiments may be set forth and described in this disclose. Some embodiments may comprise and/or define various systems, methods, articles of manufacture, apparatus, and/or devices that are either stand-alone or may be utilized together. If described as stand-alone, this does not necessarily preclude interoperability with the other disclosed embodiments. Indeed, by being included in the same disclosure, Applicant has anticipated some degree of relation between the disclosed embodiments. If described as cooperative, this does not necessarily preclude stand-alone or alternative operability. Particularly with respect to described systems, for example, while various components are described in relation to their interoperability in some embodiments, in other embodiments one or more of such components may be operative to function without the other (and/or with another component, whether disclosed or not). As such, Applicant expressly reserves the right to pursue inventive material in accordance with any differently numbered set of figures, or combinations or portions thereof, in different application filings.

This disclosure may accordingly contain multiple inventive embodiments that may individually comprise inventive material, despite being described in certain embodiments with other inventive material. Different objects disclosed in different numbered figure sets, for example, may in some cases comprise different inventive components that alone constitute the broadest extents of the disclosure herein (e.g., with or without the other different numbered figure set components). In some embodiments, the combination and/or interaction of a subset of the components may comprise inventive subject matter.

In some embodiments, each of the separate components of the BFS injection/delivery system may comprise different and/or stand-alone inventions. The BFS bottle with one or more of the described features and/or components may comprise a first invention, for example, while the BFS connection assembly with one or more of the described features and/or components thereof may comprise a second invention, and/or while the needle hub or connector with one or more of the described features and/or components thereof may comprise a third invention. In some embodiments the different inventive subject matter and/or inventions may be utilized together in one or more combined systems or configurations (which themselves may be considered different inventive combinations) while in other embodiments the different inventive subject matter and/or inventions may be utilized separately from one another. Similarly, while each component described herein is described with respect to various possible features and/or configurations, each component may exist, in some embodiments, with only a single such described feature and/or configuration. The BFS bottles described herein may, for example, include only a fluid reservoir, while the assemblies and connectors may include only such features (e.g., ports and/or threads) that may be utilized to couple to a single separate device (e.g., a BFS bottle, syringe, etc.). In some embodiments, each separate set of drawings provided with the specification may comprise a separate and/or stand-alone invention.

### IV. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

As utilized herein, the term “wing” may generally refer to any type, quantity, or configuration of exterior, axial, and/or longitudinal flange that is or becomes known or practicable. The “wing” or “wings” of a BFS vial may, for example, comprise axially elongated protrusions that extend along a portion (e.g., subset) of the length of the BFS vial. According to some embodiments, the wings may comprise flashing and/or excess plastic material that is formed along a seam of the BFS vial, e.g., formed at the joinder/intersection of the two extruded sheets of resin. In some embodiments, a wing may comprise a portion of the BFS vial(s) that is formed between mold cavities (e.g., flashing). In such embodiments, the wing may comprise a thickness equal to the compressed fusion/welding of the two plastic resin sheets. In some embodiments, a wing of a BFS vial may be at least partially formed and/or defined upon separation (e.g., via punching or cutting) of two (2) adjacently-formed BFS vial units, e.g., from a card of five (5) or more simultaneously-molded BFS vials. While the wings depicted in the figures herein are generally shown, for simplicity of illustration, as having a consistently-shaped exterior edge, in some embodiments one or more wings may comprise at least one edge feature such as a detent, protrusion (e.g., beyond the adjacent edge portions), dimples, cuts, and/or other shapes and/or features that may, for example, cooperate with corresponding portions and/or features of a hub connector (e.g., engagement tracks) to secure the wing(s) to the hub.

As used herein, the term “coupled” may generally refer to any type or configuration of coupling that is or becomes known or practicable. Coupling may be descriptive, for example, of two or more objects, devices, and/or components that are communicatively coupled, mechanically coupled, electrically coupled, and/or magnetically coupled. The term “communicatively coupled” generally refers to any type or configuration of coupling that places two or more objects, devices, components, or portions, elements, or combinations thereof in communication. Mechanical, electrical, fluid, and magnetic communications are examples of such communications. The term “mechanically coupled” generally refers to any physical binding, adherence, attachment, and/or other form of physical contact between two or more objects, devices, components, or portions, elements, or combinations thereof. The term “electrically coupled” indicates that one or more objects, devices, components, or portions, elements, or combinations thereof, are in electrical contact such that an electrical signal, pulse, or current (e.g., electrical energy) is capable of passing between the one or more objects, enabling the objects to electrically communicate with one another. In some embodiments, electrical coupling may enable electrical energy to be transmitted wirelessly between two or more objects and/or devices. The term “magnetically coupled” indicates that one or more objects, devices, components, or portions, elements, or combinations thereof, are within one or more associated magnetic fields. Objects may be electrically and/or magnetically coupled without themselves being physically attached or mechanically coupled. For example, objects may communicate electrically through various wireless forms of communication or may be within (at least partially) a magnetic field, without being physically touching or even adjacent.

References to “interior” or “exterior” are references to areas and/or portions of an object with respect to other features such as holes, volumes, ports, passages, conduits, etc. Such objects necessarily comprise and/or define various “surfaces” such as an interior, exterior, inner, outer, inside, and/or outside surface. References to the different areas and/or portions are accordingly also references to the associated surfaces.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “including”, “comprising” and variations thereof mean “including but not limited to”, unless expressly specified otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A connector assembly for coupling to a compressible vial containing a fluid agent, comprising:
a hub connector defining a longitudinal axis and having an internal chamber for at least partial reception of a neck of the vial,
wherein the hub connector includes at least one retention ledge, the at least one retention ledge configured to engage the neck of the vial to facilitate retention of the hub connector and the vial in coupled relation, and
wherein the hub connector defines a window through which at least a portion of the neck of the vial may be viewed after coupling of the hub connector to the vial;
a delivery member secured to the hub connector, the delivery member defining a subject end configured for application to a patient and an opposed vial end configured for penetrating the neck of the vial; and
at least one alignment track extending along the longitudinal axis and configured to receive a corresponding wing of the vial in secured relation therewith, to thereby couple the hub connector to the vial to enable dispensing of the fluid agent through the delivery member.

2. The connector assembly according to claim 1 wherein the vial end of the delivery member is configured to penetrate the neck of the vial during coupling of the hub connector to the vial.

3. The connector assembly according to claim 1 wherein the at least one alignment track is dimensioned to at least partially compress the wing of the vial.

4. The connector assembly according to claim 3 including first and second alignment tracks for receiving and compressing respective wings of the vial.

5. The connector assembly according to claim 4 wherein the first and second alignment tracks are disposed in diametric opposed relation.

6. The connector assembly according to claim 5 wherein each of the first and second alignment tracks includes an enclosed channel segment and an open channel segment;
wherein the wings of the vial are compressed at least within the enclosed channel segments of the first and second alignment tracks when the hub connector is coupled to the vial.

7. The connector segment according to claim 6 wherein the hub connector includes an outer wall defining axial slots therein, the open channel segments of the first and second alignment tracks comprising the axial slots;
wherein segments of the wings of the vial are at least partially received within the axial slots when the hub connector is coupled to the vial.

8. The connector assembly according to claim 2 wherein the hub connector includes at least one internal axial rib, the at least one internal axial rib configured to engage at least the neck of the vial to stabilize the vial relative to the vial end of the delivery member during coupling of the hub connector to the vial.

9. The connector assembly according to claim 8, wherein the hub connector includes a plurality of internal axial ribs.

10. The connector assembly according to claim 7, wherein the at least one internal axial rib is configured to engage at least a collar segment of the neck of the vial.

11. The connector assembly according to claim 1, wherein the at least one retention ledge is configured to engage a collar segment of the neck of the vial.

12. The connector assembly according to claim 11 wherein the hub connector includes diametrically opposed retentions ledges configured to engage the collar segment of the vial.

13. The connector assembly according to claim 12 wherein the hub connector includes internal chamfered surfaces leading to the retention ledges, the chamfered surfaces configured to facilitate passage of the collar segment of the vial through the hub connector during coupling of the hub connector to the vial.

14. The connector assembly according to claim 1, wherein the window is adjacent the at least one retention ledge.

15. The connector assembly according to claim 15 wherein the window is configured to at least partially accommodate the collar segment of the neck of the vial.

16. The connector assembly according to claim 1 including a cover positionable over the subject end of the delivery member and releasably coupled to the hub connector.

17. A connector assembly for coupling to a radially compressible vial containing a fluid agent, comprising:
a hub connector defining a longitudinal axis and having leading and trailing ends, the hub connector defining an open internal chamber for at least partial reception of a neck of the vial;
a pair of alignment tracks extending from the trailing end of the hub connector and disposed in diametric opposed relation, each alignment track including an enclosed channel segment;
one or more internal axial ribs extending along the longitudinal axis within the internal chamber of the hub connector; and
a needle secured to the hub connector, the needle defining a subject end configured for application to a subject and an opposed vial end configured for penetrating the neck of the vial;
wherein during mounting of the compressible vial to the hub connector, the neck of the vial is received within the internal chamber of the hub connector and outwardly depending opposed wings of the vial are received within the enclosed channel segments of the alignment tracks; and
wherein the one or more internal axial ribs stabilize the neck of the vial relative to the vial end of the needle to enable penetration and access to the fluid agent.

18. The connector assembly according to claim 17 wherein the hub connector includes an internal ledge, the internal ledge engaging a collar segment of the neck of the vial during longitudinal advancing movement of the compressible vial whereby, upon clearing the internal ledge, the collar segment of the vial is retained within the hub connector through engagement with the internal ledge.

19. The connector assembly according to claim 18 wherein the hub connector includes a window through which at least a portion of the neck of the vial may be viewed after coupling of the hub connector to the vial.

20. The connector assembly according to claim 19 wherein the window is configured for at least partially accommodating the collar segment of the vial.

21. A medical delivery assembly, comprising:
a vial component, including:
an outer vial wall defining a longitudinal axis and having a compressible internal chamber for storing a fluid agent;
a vial neck extending form the outer vial wall; and
one or more wings depending outwardly from the vial wall and extending along the longitudinal axis; and
a connector component, including:
a hub connector having an internal chamber for at least partial reception of the vial neck of the vial, wherein the hub connector defines a window through which at least a portion of the vial may be viewed after coupling of the hub connector to the vial;
an administration member secured to the hub connector, the administration member defining a subject end and an opposed vial end configured for penetrating the neck of the vial; and
one or more alignment tracks configured to receive the one or more wings of the vial in compressed relation therewith, to thereby couple the connector to the vial to enable dispensing of the fluid agent through the administration member.

22. The medical delivery assembly according to claim 21 wherein:
the vial includes first and second wings depending outwardly from the outer vial wall; and
the hub connector includes first and second alignment tracks configured to respectively receive the first and second wings of the vial in compressed relation therewith.

23. The medical delivery assembly according to claim 22 wherein the first and second wings are disposed in diametric opposed relation to each other and the first and second alignment tracks are disposed in diametric opposed relation.

24. The medical delivery assembly according to claim 23 wherein each of the first and second alignment tracks includes an enclosed channel segment, the first and second wings of the vial being compressed within the enclosed channel segments when the hub connector is coupled to the vial.

25. The medical delivery assembly according to claim 24 wherein the first and second wings define a wing dimension transverse to the longitudinal axis and wherein the enclosed channel segments of the first and second alignment tracks define a channel dimension transverse to the longitudinal axis, the channel dimension being less than the wing dimension such that the first and second wings are compressed within the enclosed channel segments of the first and second alignment tracks.

26. The medical delivery assembly according to claim 25 wherein each of the first and second alignment tracks includes an open channel segment;
wherein segments of the first and second wings of the vial are received within the open channel segments of the first and second alignment tracks when the hub connector is coupled to the vial.

27. The medical delivery assembly according to claim 21 wherein the neck of the vial includes a collar segment, the collar segment being engaged by a retention ledge of the hub connector when the hub connector is coupled to the vial.

28. The medical delivery assembly according to claim 27 wherein the window is adjacent to the retention ledge.

29. The medical delivery assembly according to claim 27 wherein the hub connector includes an internal chamfered surface leading to the retention ledge, the chamfered surface configured to facilitate passage of the collar segment of the vial through the hub connector during coupling of the hub connector to the vial.

30. The medical delivery assembly according to claim 27 wherein the hub connector includes multiple retentions ledges.

31. The medical delivery assembly according to claim 21 wherein the hub connector includes one or more slots, the one or more slots configured to receive the first and second wings of the vial in a first rotational orientation of the vial relative to the hub connector;
wherein, in the first rotational orientation, the opposed vial end of the administration member is spaced from the vial, the first rotational orientation corresponding to a transport condition.

32. The medical delivery assembly according to claim 31 wherein the vial is removable from the hub connector to disengage the first and second wings from the one or more slots of the hub connector to enable the hub connector and the vial to assume a second rotational orientation;
wherein, in the second rotational orientation, the first and second wings are receivable relative to the first and second alignment tracks, the second rotational orientation corresponding to an operative condition.
